(19) United States
(12) Patent Application Publication
(10) Pub. No.: US 2022/0005591 A1
(43) Pub. Date: Jan. 6, 2022

(54) METHODS FOR ADMINISTERING RESIDENTIAL CARE FACILITY

(71) Applicant: Pratibha Creative Resources LLC, Cheyenne, WY (US)

(72) Inventor: Ashish Warudkar, Palmdale, CA (US)

(21) Appl. No.: 16/920,836

(22) Filed: Jul. 6, 2020

(51) Int. Cl. G16H 40/20; G16H 10/65; G16H 50/80; G06Q 20/14; G06Q 30/00; G06Q 50/16; A61B 5/00; A61B 5/11

(52) U.S. Cl. CPC G16H 40/20; G16H 10/65; G16H 50/80; G06Q 20/14; G06Q 2230/00; G06Q 50/163; A61B 5/7275; A61B 5/746; A61B 5/1117; G06Q 30/0185

(57) ABSTRACT

Assisted living facility administration methods are disclosed for effectively preventing infection spread. A decentralized architecture and innovative systems including MedVisa™ (a certified designation of a person's health status), InfeXCON™ (a facility's real-time risk designation), a InfeXBloc™ Scorecard system (rating of facilities' implemented infection safety measures) are provided to increase resistance to infection transmission while retaining the residential setting.

### FIELD

The present invention relates to the healthcare industry, and more particularly to a senior care facility's operational management methods.

### BACKGROUND ART

Assisted living facilities, by their very nature, represent a target-rich environment for an infectious disease. Nationwide, such facilities have been heavily hit by the coronavirus pandemic. This urges us to examine our practices to improve the outcomes for our residents.

Most nursing homes and long-term care facilities weren't doing enough to protect patients from spreading infection before the coronavirus pandemic. This has been a long-term issue and so the current environment really reinforces the need to focus on these types of infection control measures.

Lockdowns in nursing and assisted living homes are not sustainable as a response to the coronavirus pandemic because the impact on seniors' psychological health due to these extended “shelter-in-place” orders is adverse.

100% testing is not an ideal solution to respond to the pandemic as well. A test result only reflects the status of that resident at that point in time. Even if all seniors were tested at any given instant, that metric can become meaningless the next day. The number of infection propagation vectors is so astronomical that one-time testing is not the entire answer. Even if periodic testing (e.g. every week) of all residents is considered, the only value that will deliver is making us aware that one or more of our residents have just tested Coronavirus positive.

A tragic combination of a virulent bug, poor administrative policies, inferior infrastructure, deficient processes, and procedures has resulted in the catastrophe. These long-term damaging effects demand action.

### SUMMARY OF THE INVENTION

The present invention specifically addresses and alleviates the above-identified deficiencies in the art. In this regard, the present invention is directed to administration methods for preventing infection spread in a residential care facility for elderly. The methods of the present invention will be exceptionally effective in providing safer environments for residents and offering new information channels for senior's family members, facility operators and public health authorities. The methods of the present invention are further operative to provide substantially more cost-effective care than prior art systems but further does not sacrifice the quality of care within the residents.

The present invention, called InfeXBloc™ architecture, encourages the senior care industry to move from an “implicit-trust” approach to a “proven-trust” infection security model. These phrases are architectural constructs and should be understood as such. In the proven-trust model, everyone is assumed “unsafe” unless proven otherwise. As a consequence, Assisted living facilities will now focus on caregiving as well as thwarting infection spread and adopt a new infection security posture that addresses the large attack surface area.

The present invention provides decentralized architectures that produce better outcomes and are more resilient. Current practice is a centralized infection management architecture, wherein hospitals are mainly doing all the work based on CDC guidance. In a distributed infection control architecture, like InfeXBloc™, facilities are empowered to take more responsibility resulting in better outcomes for seniors. Moreover, public health authorities will have improved handle on the occurrence and propagation risk of infection; the resilience of a community's overall healthcare infrastructure will be increased in the face of future outbreaks.

According to the preferred methodology, the process for administering a residential care facility to resist infection transmission comprises the initial steps of
a) unifying an entrance complex;
b) building a strong physical perimeter fence;
c) setting up an access ID key card for each entrant;
d) designating a person's health status using a MedVisa™ status granted by a nurse attesting to the healthiness of an access cardholder;
e) enforcing PPE usage;
f) setting up automated sliding doors for resident rooms and touch-free faucets;
g) designating a facility's real-time risk status using an operational InfeXCON™ status including “Green”, “Yellow” and “Red”;
h) interlinking the access ID key card, the MedVisa™ status, the automated sliding doors security system and the InfeXCON™ status to control access to each resident room;
i) video recording of all events in the facility;
j) correlating resident billing to the facility InfeXCON™ status;
k) deploying isolation rooms for residents that need isolation;
l) deploying voice activation technologies to minimize contact;
m) deploying fall alarms, and
n) deploying a scoring system—InfeXBloc™ Scorecard.

For ongoing maintenance and upgrade cycles, the process of change management further comprises of:
a) defining a stakeholder council comprising of operators, resident council members, administrators, caregivers, families, local public agencies, and Ombudsman;
b) maintaining a list of feature backlog by the administrators;
c) voting for prioritization of items on the feature backlog by the stakeholders;
d) stabilizing on a quarterly cadence for the deployment of changes;
e) providing training to all staff on changes being deployed next quarter;
informing the stakeholders when new changes are deployed;
g) soliciting feedback after a few weeks; and
h) communicating upcoming changes in the pipeline to the stakeholders in the monthly report.

Advantageously, the processes of the present invention offer safer environments to Assisted Living residents and new information channels to facility operators and health public authorities.

### BRIEF DESCRIPTION OF THE DRAWINGS

There as well as other features of the present invention will become more apparent upon reference to the drawings.

FIG. 1 shows the current centralized healthcare architecture of a community with Assisted Living homes.

FIG. 2 illustrates the decentralized healthcare architecture of a community with InfeXBloc™ assisted living homes.

FIG. 3 illustrates the operational ecosystem of an Assisted Living facility for the elderly.

FIG. 4 represents the interactions between the insiders.

FIG. 5 illustrates an implementation of strong perimeter in a proven-trust safety architecture.

FIG. 6 illustrates an implementation of maximum transmission friction in a proven-trust safety architecture.

### DETAILED DESCRIPTION OF THE EMBODIMENTS

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

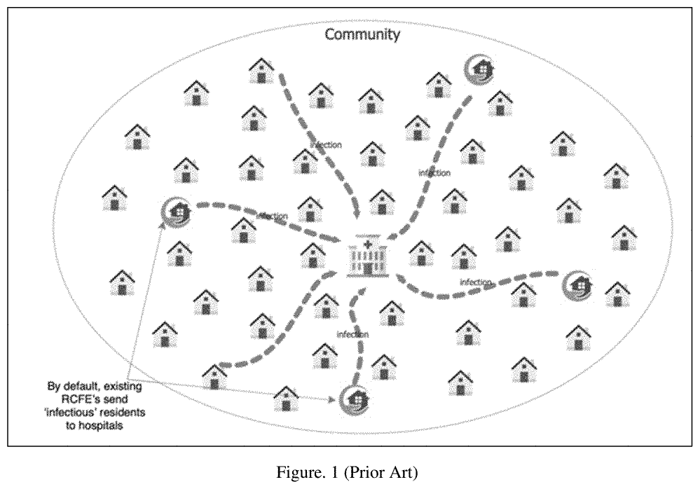

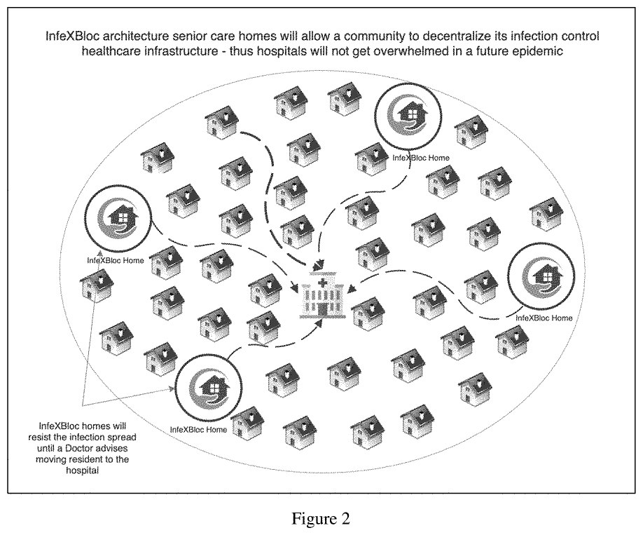

As illustrated in FIG. 1, existing healthcare architecture relies on the community hospitals to be the centralized resource for infection control. During this pandemic, this has led to overwhelming of hospitals and hence the lockdowns. By contrast, as illustrated in FIG. 2, the present invention, called InfeXBloc™ architecture, allows care homes to take up some responsibility as a decentralized infection control resource, thus lowering the worry of overwhelming. When adopted, not only will it help resist further tsunamis that can overwhelm our community hospitals, but also improve the care that Assisted Living homes can offer to their senior residents.

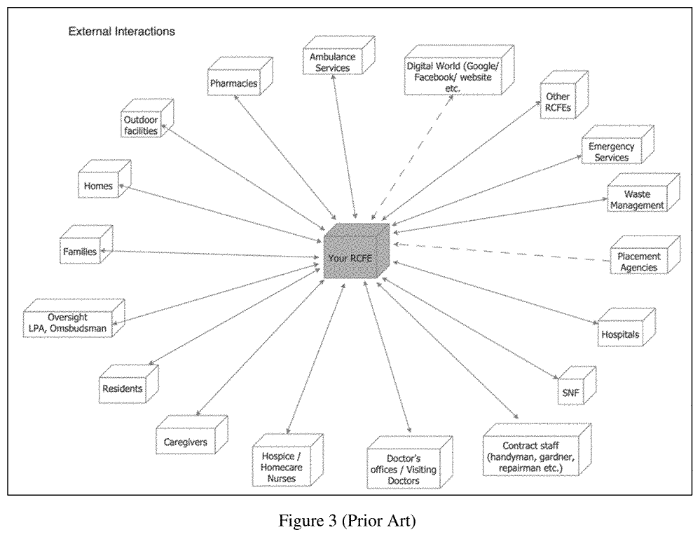

The operational ecosystem of an assisted living care facility (called RCFE in California), as depicted in FIG. 3, is complex. It is worth noting that almost all external entities are in multiples (many pharmacies, many doctors, many hospitals, etc.) and interact with the Assisted Living home multiple times every day. Borrowing terminology from the security domain, the infection attack surface area (the size and shape of the zone that exposes vulnerability) is very large.

While some industries can boast of the automatic benefits of remote working, Assisted Living homes will continue to be a hi-touch industry. While there will be opportunities to use remote examination via “tele-health visits”, the bulk of caregiving will stay hi-touch, thus requiring physical contact.

There are mainly three exposure categories that affect an assisted living home: (1) From outside-in; (2) When the bug is already inside; and (3) Transmission pathways (Via surface contact; Via aerial pathway). Each of the above exposure categories can bring infection to a uninfected senior resident.

Every interaction with the outside world represents an infection transmission vector—a pathway along which infection can travel. In FIG. 3, only the dotted lines represent interfaces that are not involved in possible infection transmission.

The number of ways in which infection can come from outside every day is:

(number of residents)×(number of visitation events from outside)

This grows geometrically as the bed capacity increases. This implies that the larger the bed capacity, the harder it is to guard against infection entering.

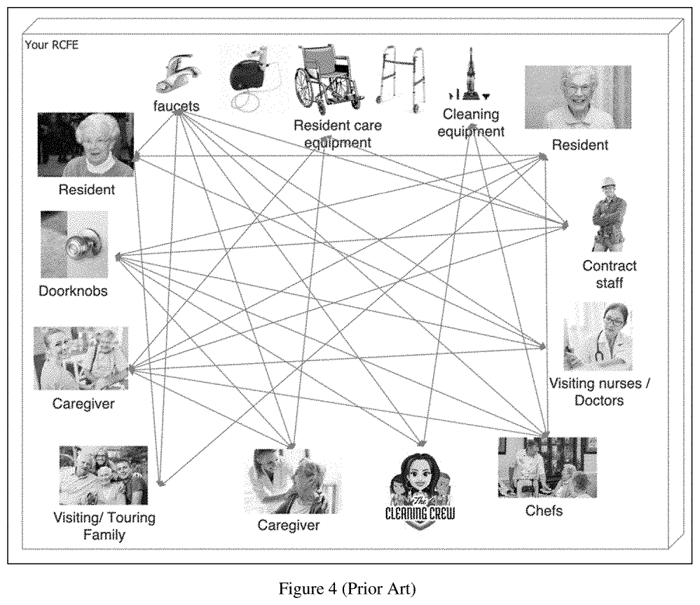

FIG. 4 represents the interactions between the insiders. On the inside, there are two types of actors: human (residents, caregivers, visiting professionals, staff, contract workers, family members, etc.) and non-human (care equipment like wheelchairs and walkers, cleaning equipment, faucets, doorknobs, etc.). Every interaction represents another infection transmission vector. Like before, each entity is in multiples. Inside the assisted living home, the attack surface area grows exponentially. This problem gets compounded because in closed spaces, like an assisted living home, the HVAC system recirculates air and typical HVAC systems are not designed to disinfect. As a result, this makes the attack surface area hyper large and any human actor can be the source and anyone in the vicinity can be a target.

While in real life, completely sterile spaces may only be found in cleanrooms for surgery, NASA labs or pharmaceutical manufacturing, given the congregation of seniors (some of them may even be immuno-compromised), the only way to deal with a highly corrupted domain is by introducing friction along the transmission pathways. The present invention is directed to methods for resisting infection spread in assisted living homes, comprising steps to introduce friction in the pathway of the spread: minimizing touch to surfaces; using alcohol-based disinfectants; washing hands; and changing gloves; facemasks, face shields, gowns at appropriate intervals.

The current operating model before March 2020 was implicit-trust based. Everyone was “safe” unless they proved otherwise. Facility infrastructure was more directed to caregiving needs, e.g., activities of daily living, fall prevention features like grab bars in corridors and bathrooms, non-slippery ambulatory surfaces, clutter-free and spacious rooms, etc.

With InfeXBloc™, we implement the proven-trust policy, the assumption that everyone is safe inside the assisted living home is rejected and the danger of the bug being present inside is embraced. Thus, everyone is “unsafe” unless proven otherwise. As a consequence, Assisted Living home must now focus on caregiving as well as on thwarting infection spread and must adopt a new infection security posture that addresses the hyper large attack surface area.

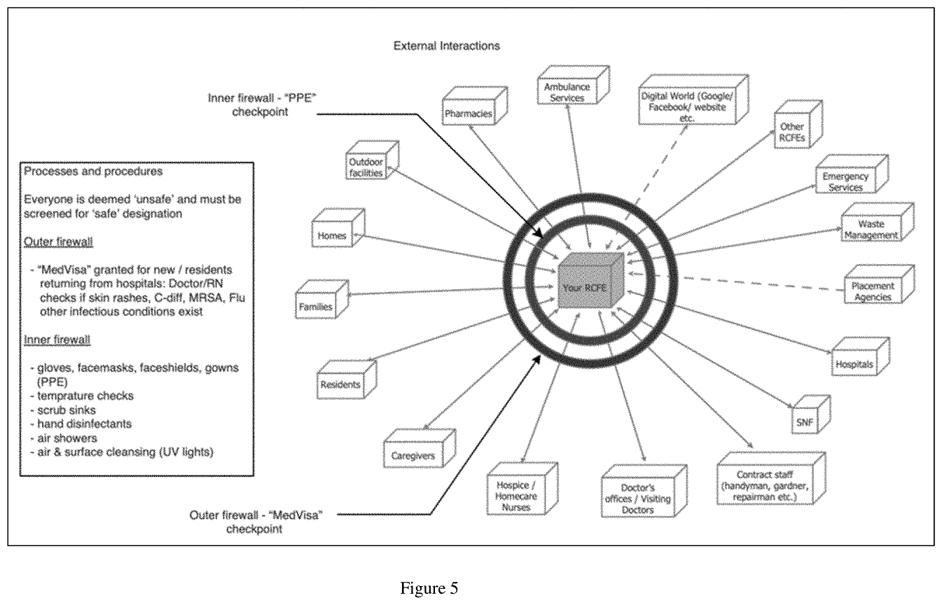

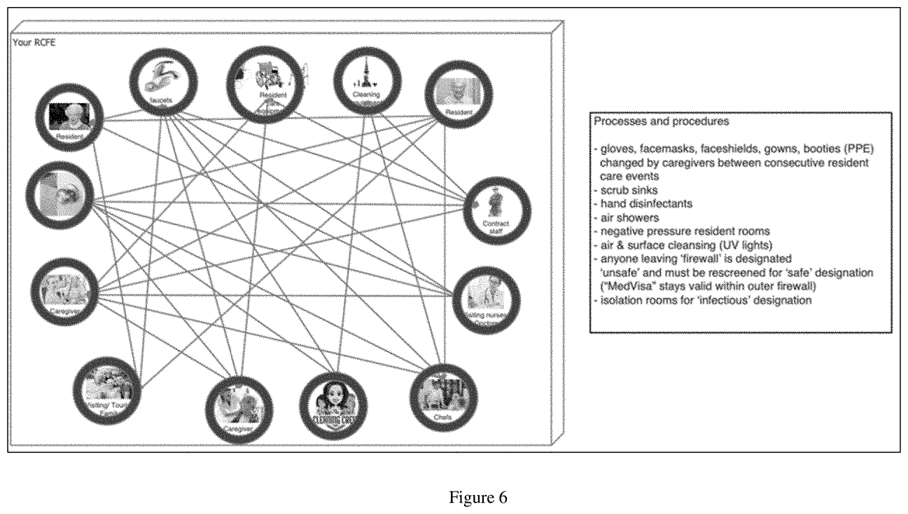

The present invention is directed to a proven-trust safety architecture comprising: (1) implementation of strong perimeter (FIG. 5); and (2) implementation of maximum transmission friction (FIG. 6).

Conceptually, the idea illustrated in FIG. 6 is that each resident's room defends itself. Maximize resistance to infection transmission within the facility is created in this way. This will be done by adopting many procedures designed to thwart cross-transmission inside the facility. An operational assisted living facility represents a micro-community of human and non-human actors with residents, caregivers, visiting families, caregiving equipment, etc. When each contact between these actors is sandwiched between disinfecting steps the resistance for infection transmission is greatly increased. This will be achieved by the use of several: consumables (disinfectants, gloves, face masks, face shields, protective gowns, etc.); equipment (touch-free doors, touch-free faucets, UV lights, negative air pressure HVAC, etc.); processes (single entrance gate, resident room doors that can require caregivers to swipe access cards, check if a caregiver has her face mask and gloves on, etc.); and procedures (a robust audit trail of events to establish adherence to protocol).

Referring to FIG. 5, everyone is deemed “unsafe” and must be screened for “safe” designation. There will be two firewalls to prevent infectious spread. The outer firewall implements “MedVisa™” granted for new residents or residents returning from hospital. A licensed professional (Doctors/RN etc.) may check if there are any skin rashes, flu, MRSA, any other infectious conditions prior to granting “MedVisa™”.

In some embodiments, a program called “MedVisa™” is implemented to designate a person's health status. This may manifest in two ways: (1) a licensed practitioner (Doctor, PA, RN) screens a new resident/returning resident from a hospital stay and certifies him as infection-free. A lighter version of it will be for non-residents (family or guest visitors) who may closely interact with the residents (outer firewall in FIG. 5); and (2) Both the above will be accompanied by an inner firewall (inside ring in FIG. 5). MedVisa™ is an effective medical clearance informing that the entrant into an Assisted Living home is safe.

A MedVisa™ is a medical certification attesting to the healthiness of the access cardholder. The MedVisa™ granted or denied by a nurse at the entrance (for residents, caregivers, volunteers, activity staff, etc.); self-certified by visiting doctors, RNs, LVNs (grouped as ‘credentialed professionals’); employer certified for licensed contract staff or employees of delivery companies. The Key card will be surrendered when the holder leaves the facility premises. If a holder of the access card develops unsafe medical symptoms while on premises, his access will be revoked, and he will be escorted outside the entrance complex.

In some embodiments, a program called “InfeXCON™” is implemented to designate the facility's real-time risk status. The facility will be designated with an operational InfeXCON™ status including “Green” that implies no residents are designated infectious; “Yellow” that implies one or more residents are designated infectious and have been quarantined in their rooms with negative pressure HVAC; and “Red” that implies one or more residents with a high severity infectious disease (like coronavirus infection) present and isolation rooms have been deployed.

The facility's InfeXCON™ status will have an impact on the severity of infection security protocols being enforced. For example, green status implies resident room doors are open, socialization is free, lunch and dinners are being served in common dining areas, activities are happening, etc. Yellow status implies restrictions are in play, and family visitation is constrained. Red status implies very strict controls are in play, and isolation rooms are being used. InfeXCON™ is a transient status and can be changed in either direction based on a licensed practitioner's decision.

According to one embodiment, the resident's billing may be correlated to the facility InfeXCON™ status. The pricing structure will factor in the InfeXCON™ status as follows: base pricing will apply assuming InfeXCON™ is Green and incremental pricing will apply when InfeXCON™ designation is turned Yellow or Red. Everyday morning a text message will be sent to the resident's family member's phone regarding the InfeXCON™ status. This will also be sent when the InfeXCON™ status is changed.

In some embodiments, a scoring program called “InfeXBloc™ Scorecard” is implemented for developing an objective measurement system that can be used to assess infection control effectiveness. This will be a balanced scorecard that incorporates multiple assessment criteria. The assessment criteria comprises ingress control, PPE preparedness, transmission resistance processes, disinfection protocols, isolation rooms, and family-friendly access protocols. The InfeXBloc™ Scorecard will help placement agencies to gauge whether a facility is suitable for their clients. The InfeXBloc™ Scorecard will allow insurers to have a codified quantifiable measure to define their underwriting criteria, and objectively correlate to the insurance premiums. Such codification can then be extended to track the number of infections originating at the facility. This can be used as an ongoing measure to assess performance.

The present invention is directed to methods for implementing InfeXBloc™ architecture, comprising:
a) unifying an entrance complex;
b) building a strong physical perimeter fence;
c) setting up an access ID key card for each entrant,
d) designating a person's health status using MedVisa™ which becomes part of the key;
e) enforcing PPE usage;
f) setting up automated sliding doors for resident rooms and touch-free faucets;
g) designating InfeXCON™ status for the facility;
h) Interlinking the ID key card, the MedVisa™ status, the automated sliding doors security system and the InfeXCON™ designation to control access to each critical resource (resident rooms);
i) Deploying the security principle of ‘least privilege’;
j) Video recording of all events in the facility;
k) Correlating the resident billing to the facility InfeXCON™ status;
l) Deploying isolation rooms;
m) Deploying fall alarms; and
n) Deploying a scoring system—InfeXBloc™ Scorecard.

A singular entrance complex will be used for the following purposes: controlling access, validating authentication and authorization; providing a Nurse to grant or deny MedVisa™; providing for change of street clothes into facility provided scrubs; providing thermal cameras for temperature sensing; receipt of deliveries from suppliers; and providing temporary exit-passes and infection-controlled breakroom for caregivers

Each entrant will be provided a magnetic stripe key card. This card will contain data points comprising user's identification; MedVisa™ status; authorization privileges that implement the least-privilege pattern; and facility InfeXCON™ designation.

Every resident room will have a scanner outside which can scan for the N-95 face mask, gloves, other PPE and allow or disallow the opening of the sliding door when the key card is swiped.

Equipment and technologies to implement the InfeXBloc™ architecture may include: negative pressure HVAC systems with high air changes per hour (OSHPD standard for isolation rooms is 12 ACH); anterooms that can allow caregivers to discard PPE after a care event for an “infectious” resident; directing airflow from ceiling to resident's bed to exhaust vent; returning exhaust from a resident room should not mix with clean air going to the room; air scrubbing/cleaning within the room (e.g. SAM400 from Scientific Air Management).

The present invention for implementing change management of InfeXBloc™ architecture, further comprising:
a) defining a stakeholder council comprising of operators, resident council members, administrators, caregivers, families, LPA, Ombudsman;
b) maintaining a list of feature backlog by the administrator;
c) voting for prioritization of items on the backlog by stakeholders;
d) stabilizing on a quarterly cadence for the deployment of changes;
e) providing training to all staff on changes being deployed next quarter;
f) informing stakeholders when new changes are deployed;
g) soliciting feedback after a few weeks; and
h) communicating upcoming changes in the pipeline to all stakeholders in the monthly report.

The physical architecture for implementing InfeXBloc™ architecture comprises:
a) a strong perimeter entrance gate, an entrance temperature check station, a caregiver changing room for changing from street clothes to facility cleaned scrubs, donning of PPEs, the deposit of cell phones, etc.;
b) design of rooms for “infection containment” (negative pressure HVAC, touch-free door, touch-free faucets, private baths as far as possible, anterooms, the ability for caregivers to discard biohazardous waste, air scrubbing equipment, etc.);
c) possible design of sunroom—as this has disinfectant qualities and is beneficial for seniors;
d) hands-free sliding room doors to minimize contact surfaces;
e) hands-free faucets that automatically activate/deactivate to minimize contact surfaces;
f) shower handles, toilet flush handles, doorbells should be cleansed/disinfected regularly;
g) pressure-indicator outside of rooms will indicate if the room is negative or positive pressure so that the caregivers can determine the course of entry;
h) scrubbing stations in rooms as well as for a collection of rooms;
i) UVC lights for room disinfection; and
j) cleaning and disinfecting wheelchairs, walkers, canes, gurneys, blood pressure monitors, thermometers, medicine carts, nurse's iPad, and other care equipment; stairs, banisters, elevator components, furniture, and other facility fixtures; and books in the library.

The process design for implementing InfeXBloc™ architecture comprises:
a) verification/validation/medical check for granting of MedVisa™;
b) mandatory hand washing between every caregiving event;
c) residents with an infectious sickness will not leave their rooms and will be provided with meals in the room;
d) no bags permitted past the outer firewall;
e) facility will not allow street footwear, but will provide disinfected house shoes for everyone except non-caregivers who must wear work boots as part of OSHA compliance. These actors will be provided booties;
f) groceries, vegetables, meats, fruits, supplies, bags, and similar items will be cleaned and disinfected before storage;
g) schedule rooms/common areas will be vacant for one hour per day for UVC disinfection; and
h) laundry will be done once a day separately for each resident to avoid cross-contamination amongst residents' laundry.

Resident's families must be educated about the shared responsibility of placing their beloved senior in an InfeXBloc™ home in the following ways:
a) visiting family members must obtain MedVisa™ screening, wear appropriate PPE, booties, etc.;
b) physical contact with residents will only be across plexiglass dividers or “hug-screens” if the facility is not in Green status;
c) give a scoring card (InfeXBloc™ Scorecard) for resident families to score a facility;
d) encourage virtual visits if the facility is not in Green status;
e) weekly video report posted on the website which will include medication journal, care journal, etc. for each resident (this is based on resident granting HIPAA permissions);
f) family members can have authenticated access to each resident's webpage on the facility website;
g) each resident's belongings (physical and digital) will be part of a farewell package delivered to the family after resident's death.

Placement agencies may use the InfeXBloc™ Scorecard to gauge if a facility is suitable for their clients and encourage prospective residents to use a virtual 3D tour of the facility to get a preview.

Residents in a InfeXBloc™ facility should expect some restrictions to mobility when they are diagnosed with infectious diseases unless the Doctor designates them as needing isolation, in which case they will be relocated to “isolation” rooms (if such a room is available, else they will be transferred to a hospital). Residents must use face masks when interacting with other residents. Residents should be outside of their room when the UVC light/Robotic disinfecting will be performed. Residents will expect a stronger cleaning and disinfection routine and enjoy more connectedness via virtual visits. Resident's meetings with family members will adhere to ‘social distancing’ norms until normal operations resume.

Doctors, Nurses, and other health professionals in a InfeXBloc™ facility can self-certify their MedVisa™. They should follow the PPE protocols and should encourage virtual health visits.

Non-caregiving staff including chefs, handymen, equipment maintenance staff, hairdressers, delivery staff, and other non-caregiving staff should enter only via “entrance complex”. They must sign a self-declaration to obtain MedVisa™ on site and avoid resident contact unless essential.

Regulators, inspection agents, ombudsman and similar individuals will be enabled for virtual visitation and virtual inspection. They will have access to monthly ‘State-of-Assisted-Living-home’ video reports and are able to use conference rooms for personnel interviews, audit reviews, etc.

Volunteers must sign a self-declaration to obtain MedVisa™ on site.

Activity staff must sign a self-declaration to obtain MedVisa™ on site and avoid contact with residents.

The delivery staff will have their company certify their MedVisa™ and have minimal contact with residents.

The cost of care is expected to be more for InfeXBloc™ architecture than before InfeXBloc™, given the strenuous extra work and responsibility assumed. However, InfeXBloc™ will endeavor to provide a facility that significantly restricts an infection from entering inside the facility and from spreading by transmission inside the facility even if a resident is designated ‘infectious’.

The InfeXBloc™ architecture will offer tremendous benefits for all stakeholders involved. Residents and families will feel safer after the coronavirus pandemic. Residents and families will have real-time visibility into the InfeXCON™ status of the facility that their loved one is living in. After the facility downgrades its InfeXCON™ status, the collective stress relief will be priceless.

Single facility operators can provide safer environments for residents and will have higher personal satisfaction that they have done the best for their residents. Single facility operators that are not using the InfeXBloc™ architecture, and have a resident who contracts an infectious disease are currently obliged to transfer the infected resident to a hospital. If there is an InfeXBloc™ facility with an isolation room nearby, the single facility could consider transferring the resident to that facility based on some mutual contractual agreement. If all the resident needs is quarantine and not critical care, local quarantine in the facility rather than relying on a hospital offers several advantages. Other residents are protected. The infectious resident is ensured to get the quarantine care he deserves. The infectious resident is protected from having exposure to a hospital, which tends to be a higher risk facility. The Medicare/Medicaid cost of care at an InfeXBloc™ facility can be lower than a hospital stay. It prevents hospital overcrowding from non-threatening situations. At the end of the quarantine period, the resident can be transferred back to the original Assisted Living home.

Multi-facility operators can provide safer environments for residents. They can make informed decisions about deploying shared resources across owned facilities (e.g., Should their caregiver who works fulltime in ‘Facility A’ work overtime in ‘Facility B’, if Facility B is operating at InfeXCON™ level ‘Yellow’/′Red′?). These operators have personal satisfaction that they have done the best for their residents.

When InfeXBloc™ architecture gets adopted at scale, Department of Social Services (DSS) can know in real-time if a significant number of facilities in a geographical area are turning from “Green” to “Yellow”, indicating an infection trend. Such real-time visibility will be priceless and can allow DSS to share alerts across the geographical area and share expert recommendations to facilities in that area or mobilize resources, if necessary. The value of such a real-time dashboard can be priceless and can allow DSS to deal with a fast or silently spreading infection and thus thwart an impending crisis. As a result, the hospital infrastructure will be better protected from any future surges of demand due to infection spread.

A caregiver's career path is enriched when he/she has on-the-job education and experience in serving in InfeXBloc™ facilities. Everyone in direct caregiving (Caregivers, Med-techs, Nurses, Doctors, etc.) will have a sense of fulfillment when he/she can meaningfully serve a resident(s) who may be experiencing infections with a lower risk of contracting the disease themselves. This can contribute to lowering and ultimately eliminating any stigma associated.

For insurance companies, the InfeXBloc™ Scorecard will allow insurers to have a codified quantifiable measure to define their underwriting criteria, and objectively correlate to the insurance premiums. Such codification can then be extended to track the number of infections originating at the facility. This can be used as an ongoing measure to assess performance.

The implementation of the present InfeXBloc™ architecture will substantially decrease the number of the infection transmission vectors that get activated in the daily care of the one resident in the care facility. For instance, if each resident needs a care visit by a caregiver once in two hours, the care giver visits the resident's room 12 times in total daily. For each visit, the caregiver opens and closes the room door twice (once during entry and next during exit). Thus, daily the number of touches that caregiver has with the doorknobs is 48 times. If each resident leaves room for breakfast, lunch, dinner, and one additional activity, the number of time the resident may interact with the doorknobs is 16 times daily. If each resident uses the restroom once in 3 hours, the daily number of touches is 32. If the caregiver is assisting in restroom or showering activities, these number of interactions may be executed by the caregiver. It is also assumed that each resident uses faucets 16 times daily, flushes toilets 8 times daily, and uses grab bars 8 times daily. Collectively, the total number of the infection transmission vectors that get activated in the daily care of the one resident in the care facility is 128 times. If the facility had 20 beds, the total number of active infection transmission vectors would be 20×128=2560.

In the event that one resident in the InfeXBloc™ facility becomes infectious, the InfeXCON™ facility status will go “Yellow” and that resident's room will deploy the negative HVAC features and all doors will be closed automatically. From that point, doors will open in a touch free mode (nicknamed “Star Trek” doors). Thus, by designating InfeXCON™ status as “Yellow”, the number of active infection vectors in the care of the other 19 residents can be reduced.

Table 1 shows lowered cross-transmission risk by deploying tough-free technologies. If a 6:1 ratio of residents to caregivers is assumed in the facility, the tough-free technologies would better protect the infectious resident from the other 19 residents by lowering the cross-transmission possibilities by 83.3% (640 infection vectors isolated from a total of 768 infection vectors) and protect the 19 healthy residents from the infectious resident by lowering the cross-transmission possibilities by 16.6% (128 infection vectors isolated from a total of 768 vectors). If a 12:1 ratio of residents to caregivers is assumed, the tough-free technologies would better protect the infectious resident from the other 19 residents by lowering the cross-transmission possibilities by 91.6% (1408 infection vectors isolated from a total of 1536 infection vectors) and protect the 19 healthy residents from the infectious resident by lowering the cross-transmission possibilities by 8.3% (128 infection vectors isolated from a total of 1536 infection vectors). If a 20:1 ratio of residents to caregivers is assumed, the tough-free technologies would better protect the infectious resident from the other 19 residents by lowering cross-transmission possibilities by 95% (2432 infection vectors isolated from a total of 2560 infection vectors) and protect the 19 healthy residents from the infectious resident by lowering the cross-transmission possibilities by 5% (128 infection vectors isolated from a total of 2560 infection vectors). It is important to note that the extent of risk reduction is related to the caregiver to resident ratio. The higher this ratio, the risk reduction is different.

TABLE 1
Lowering of cross-transmission risk
by deploying touch-free technologies

| | Lowered risk of cross- | Lowered risk of cross- |
|---|---|---|
| Caregiver to | transmission for Infectious | transmission for healthy |
| resident ratio | resident | residents |
| 6:1 | 83.3% | 16.6% |
| 12:1 | 91.6% | 8.3% |
| 20:1 | 95% | 5% |

Tabulating the results, it can be concluded that deploying the tough-free technologies can significantly dampen the cross-transmission possibilities. Moreover, the resident to caregiver ratio impacts the two populations differently. Deploying the tough-free technologies lowers the risk for the infectious resident better when caregiver to resident ratio is higher, but the healthier residents get lesser protection. Hence deployment of the tough-free technologies is more beneficial to the infectious resident. Deploying the tough-free technologies lowers the risk for healthier residents more when caregiver to resident ratio is lower, but the infectious resident get lesser protection. Hence deployment of the tough-free technologies is more beneficial to the healthier residents. In either case, deployment of multiple technologies will further reduce the extent of infection cross-transmission vectors invoked.

It is important to note that when we go from one infectious resident to two or more infectious residents, the degree of risk lowering stays the same. This is because by adopting the “proven trust” architecture, a “micro-segmentation” has been implemented. From an infection security perspective, in existing assisted living homes, the blast radius is the entire assisted living home facility. By contrast, in the InfeXBloc™ home, the blast-radius has been shrunk to a single resident room. Thus the facility attains maximum achievable cross-transmission resistance at the first appearance of an infection and any additional resident becoming infectious does not raise the threat for the healthier ones any higher.

Based on the above quantitative analysis, the adoption of the InfeXBloc™ architecture provide safer environment for an assisted living facility.

1.-5. (canceled)

6. A system for administering a residential care facility to resist infection transmission, comprising:
a magnetic stripe access ID key card for each entrant of the facility, wherein the key card contains data comprising an access cardholder's identification and health status granted by a nurse attesting to healthiness of the access cardholder;
a real-time dashboard showing facility's real-time risk status, wherein the risk status has an impact on a severity of infection security protocols to be enforced, and wherein the risk status is correlated to resident billing, and wherein the real-time dashboard is configured to allow Department of Social Service to share alerts across a geographic area and share expert recommendations to the facility; and
a scoring program deploying a scoring system to quantify infection control effectiveness in the facility, wherein the scoring program generates a score based on ingress control, PPE preparedness, transmission resistance processes, disinfection protocols, isolation rooms, and family-friendly access protocols in the facility, and wherein the score is correlated to insurance premiums.

7. The system of claim 6, wherein the facility's real-time risk status comprises a “Green” status implying free socialization, a “Yellow” status implying restricted socialization and constrained family visitation, and a “Red” status implying strict control and isolation rooms being used.

8. A method for administering a residential care facility to resist infection transmission, comprising:
setting up a magnetic stripe access ID key card for each entrant of the facility, wherein the key card contains data comprising an access cardholder's identification and health status granted by a nurse attesting to healthiness of the access cardholder;
designating, via a real-time dashboard, a facility's real-time risk status, wherein the risk status has an impact on a severity of infection security protocols to be enforced, wherein the risk status is correlated to resident billing, and wherein the real-time dashboard is configured to allow Department of Social Service to share alerts across a geographic area and share expert recommendations to the facility;
deploying a scoring program to quantify infection control effectiveness in the facility, wherein the scoring program generates a score based on ingress control, PPE preparedness, transmission resistance processes, disinfection protocols, isolation rooms, and family-friendly access protocols in the facility, and wherein the score is correlated to insurance premiums; and
implementing touch-free facilities in the facility.

9. The method of claim 8, wherein the facility's real-time risk status comprises a “Green” status implying free socialization, a “Yellow” status implying restricted socialization and constrained family visitation, and a “Red” status implying strict control and isolation rooms being used.
